(19) United States
(12) Patent Application Publication
(10) Pub. No.: US 2025/0226782 A1
(43) Pub. Date: Jul. 10, 2025

(54) ACTIVE VIBRATION CONTROL SYSTEM

(71) Applicant: LORD CORPORATION, Cleveland, OH (US)

(72) Inventors: Lee Wakefield, Holly Springs, NC (US); Anthony Hunter, Raleigh, NC (US); Douglas Swanson, Cary, NC (US); Sean Coots, Durham, NC (US)

(21) Appl. No.: 18/848,559

(22) PCT Filed: Mar. 23, 2023
(86) PCT No.: PCT/US2023/016106

§ 371 Date: Sep. 19, 2024

Related U.S. Application Data
(60) Provisional application No. 63/222,858, filed on Jul. 16, 2021.

(51) Int. Cl. H02P 25/032; B64D 31/16; B64D 45/00; H02K 7/06

(52) U.S. Cl. CPC H02P 25/032; B64D 31/16; B64D 45/00; H02K 7/063; B64D 2045/0085

(57) ABSTRACT

This disclosure provides a circular force generator system (100) which incorporates a failsafe component. The failsafe component provides a brake system on the circular force generator such that the rotor of the circular force generator is rapidly brought to a stop in response to a predetermined sensor output or a manual override of the circular force generator system.

### CROSS REFERENCE TO RELATED APPLICATIONS

The present application claims priority to U.S. Provisional Application No. 63/322,858 filed on Mar. 23, 2022 which is incorporated herein.

### BACKGROUND

Fixed wing and rotary wing aircraft frequently experience unwanted vibrations within in the airframe of the aircraft. Circular force generators (CFGs) have been used to mute or at least reduce these unwanted vibrations. However, the systems which manage the CFGs can fail or operate in a manner which also produce dangerous vibrations. Such failures can take the form of:
rotor underspeed or overspeed leading to unsafe airframe resonance;
CFG force magnitude greater than desired leading to airframe or CFG damage;
CFG force phase is incorrect leading to increased airframe vibrations.

Additionally, current CFG systems do not provide for a controlled shut down of the CFG rotors. In the current CFG systems, the CFG rotors freewheel until friction brings the rotors to a stop. During this time, aircraft vibrations may “reactivate” the rotors producing a “hula-hoop” effect and leading to an increase in airframe vibrations. The following disclosure provides improvements to the operation and design of the CFG systems which overcome the identified shortcomings and also provides for rapid braking of CFG rotors to a safe speed or even a full stop.

FIG. 1 depicts a conventional prior art CFG system 01. CFG system 01 has three primary components: a circular force generator (CFG) 5; a command circuit 30; and, a monitoring system 50. CFG 5 may have multiple rotors with each rotor supporting a mass. Command circuit 30 provides primary control over CFG 5 while monitoring system 50 provides safety control of CFG 5. CFG 5 includes at least one electric motor 10. Command circuit 30 includes a force command digital bus 41, a command processor 32, speed and/or position sensors 36a, a control power source 38, an electrical circuit 39, electromagnetic interference (EMI) filter with lightning protection 43, a low voltage control power supply 37 and a motor driver 34. Monitoring system 50 includes a second power supply identified as motor power 52, an electrical circuit 53, a monitor processor 54, a vibration sensor 57, speed and/or position sensors 36b. Electric current from motor power 52 passes through a second EMI filter with lightning protection 63 to motor driver 34. Monitoring system 50 provides safety control of 5 by interrupting flow of electric current to motor driver 34 by opening of relay 56 when an unsafe condition occurs (force, vibration, speed are erroneous as determined by the monitor processor and vibration, speed and/or position sensors).

### SUMMARY

In one aspect, the present disclosure describes a circular force generator system (CFG system) comprising an electric motor, the electric motor having a rotor, a stator core, primary windings and a braking coil. The rotor supports a mass which rotates with the rotation of the rotor. The CFG system includes a command circuit and a monitoring system. The command circuit includes a command processor and a sensor for monitoring rotational speed and/or radial position of the mass or rotor with the sensor in electronic communication with the command processor. The command circuit also includes a first power supply providing electrical current to the electric motor via a first electrical circuit. The monitoring system includes a monitor processor which controls a first relay. During operation of the CFG system, the first relay is closed and is positioned within the first electrical circuit. The monitor processor provides control over the first relay such that opening of the first relay by the monitor processor removes electrical current from the electric motor. During operation of the CFG system, a second relay is open within a second electrical circuit (159). The monitor processor provides control over the second relay and closure of the second relay activates the braking coil. The monitoring system may also include a vibration sensor which provides data to the monitor processor.

In one aspect, the present disclosure describes a circular force generator system (CFG system) comprising an electric motor, the electric motor having a rotor, a stator core, primary windings and a braking coil. The rotor supports a mass which rotates with the rotation of the rotor. The CFG system includes a command circuit and a monitoring system. The command circuit includes a command processor and a sensor for monitoring rotational speed and/or radial position of the mass or rotor with the sensor in electronic communication with the command processor. The command circuit also includes a first power supply providing electrical current to the electric motor via a first electrical circuit. The monitoring system includes a monitor processor which controls a first relay and a second relay. During operation of the CFG system, the first relay is closed and is positioned within the first electrical circuit. During operation of the CFG system, the second relay is open within a second circuit. The monitoring system also includes a second sensor which monitors rotational speed and/or radial position of the mass or rotor with the second sensor in electronic communication with the monitor processor. The monitor processor provides control over the first relay such that opening of the first relay by the monitor processor removes electrical current from the electric motor. The monitor processor also provides control over the second relay and closure of the second relay activates the braking coil. The monitoring system may also include a vibration sensor which provides data to the monitor processor.

In one aspect, the present disclosure describes a circular force generator system (CFG system) comprising an electric motor, the electric motor having a rotor, a stator core, primary windings and a braking coil. The rotor supports a mass which rotates with the rotation of the rotor. The CFG system includes a command circuit and a monitoring system. The command circuit includes a command processor and a motor driver in electronic communication with the command processor. The command circuit also includes a sensor for monitoring rotational speed and/or radial position of the mass or rotor with the sensor in electronic communication with the command processor. The command circuit also includes a first power supply providing electrical current to the electric motor via a first electrical circuit. The monitoring system includes a second power supply which supplies electrical current to the motor driver via a second electrical circuit. The monitoring system also includes a monitor processor which controls a first relay and a second relay. During operation of the CFG system, the first relay is closed and is positioned within the first electrical circuit. During operation of the CFG system, the second relay is closed and is positioned within a second circuit. The monitoring system also includes a second sensor which monitors rotational speed and/or radial position of the mass or rotor with the second sensor in electronic communication with the monitor processor. The monitoring system also includes a third open relay positioned within a third electrical circuit. The monitor processor provides control over the first and second relays such that opening of the first and second relays by the monitor processor removes electrical current from the electric motor. The monitor processor also provides control over the third relay and closure of the third relay activates the braking coil. The monitoring system may also include a vibration sensor which provides data to the monitor processor.

In another aspect, the present disclosure provides a method for controlling a force generator system (CFG system). The CFG system includes an electric motor, the electric motor having a rotor, a stator core, primary windings and a braking coil. The rotor supports a mass which rotates with the rotation of the rotor. The CFG system includes a command circuit and a monitoring system. The command circuit includes a command processor and a sensor for monitoring rotational speed and/or radial position of the mass or rotor with the sensor in electronic communication with the command processor. The command circuit also includes a first power supply providing electrical current to the electric motor via a first electrical circuit. The monitoring system includes a monitor processor which controls a first relay. During operation of the CFG system, the first relay is closed and is positioned within the first electrical circuit. The monitor processor provides control over the first relay such that opening of the first relay by the monitor processor removes electrical current from the electric motor. A second open relay is positioned within a second electrical circuit (159). The monitor processor provides control over the second relay and closure of the second relay activates the braking coil. The method operates the CFG system by rotating the rotor and mass while using the sensor to monitor rotational speed and/or radial position of the mass and/or rotor. The monitoring system may also include a vibration sensor which provides data to the monitor processor. The command processor calculates a force generated by the mass as the mass and rotor rotates and the command processor manages electrical current to the electric motor. The monitor processor interprets data received from the sensor and energizes the braking coil by closing the second relay when the data from the sensor indicates an out of range condition for the rotational speed or position of the mass or rotor position. The monitor processor may also interpret data from the vibration sensor and energize the braking coil in response to the interpreted data.

In another aspect, the present disclosure provides a method for controlling a force generator system (CFG system). The CFG system includes an electric motor, the electric motor having a rotor, a stator core, primary windings and a braking coil. The rotor supports a mass which rotates with the rotation of the rotor. The CFG system includes a command circuit and a monitoring system. The command circuit includes a command processor and a sensor for monitoring rotational speed and/or radial position of the mass or rotor with the sensor in electronic communication with the command processor. The command circuit also includes a first power supply providing electrical current to the electric motor via a first electrical circuit. The monitoring system includes a monitor processor which controls a first relay and a second relay. The first relay is closed and is positioned within the first electrical circuit. The second relay is open and is positioned within a second circuit. The monitoring system also includes a second sensor which monitors rotational speed and/or radial position of the mass or rotor with the second sensor in electronic communication with the monitor processor. The monitor processor provides control over the first relay such that opening of the first relay by the monitor processor removes electrical current from the electric motor. The monitor processor provides control over the second relay and closure of the second relay activates the braking coil. The monitoring system may also include a vibration sensor which provides data to the monitor processor. The method operates the CFG system by rotating the rotor and mass while using the first and second sensors to monitor rotational speed and/or radial position of the mass and/or rotor. The command processor calculates a force generated by the mass as the mass and rotor rotates and the command processor manages electrical current to the electric motor. The monitor processor to interprets data received from the second sensor and energizes the braking coil when the data from the second sensor indicates an out of range condition for the rotational speed or position of the mass or rotor position. The monitor processor may also interpret data from the vibration sensor and energize the braking coil in response to the interpreted data.

In another aspect, the present disclosure provides a method for controlling a force generator system (CFG system). The CFG system includes an electric motor, the electric motor having a rotor, a stator core, primary windings and a braking coil. The rotor supports a mass which rotates with the rotation of the rotor. The CFG system includes a command circuit and a monitoring system. The command circuit includes a command processor and a motor driver in electronic communication with the command processor. The command circuit also includes a sensor for monitoring rotational speed and/or radial position of the mass or rotor with the sensor in electronic communication with the command processor. The command circuit also includes a first power supply providing electrical current to the electric motor via a first electrical circuit. The monitoring system includes a second power supply which supplies electrical current to the motor driver via a second electrical circuit. The monitoring system also includes a monitor processor which controls a first relay and a second relay. The first relay is closed and is positioned within the first electrical circuit. The second relay is closed and is positioned within a second circuit. The monitoring system also includes a second sensor which monitors rotational speed and/or radial position of the mass or rotor with the second sensor in electronic communication with the monitor processor. The monitoring system also includes a third open relay positioned within a third electrical circuit. The monitor processor provides control over the first and second relays such that opening of the first and second relays by the monitor processor removes electrical current from the electric motor. The monitor processor provides control over the third relay and closure of the third relay activates the braking coil. The monitoring system may also include a vibration sensor which provides data to the monitor processor. The method operates the CFG system by rotating the rotor and mass while using the first and second sensors to monitor rotational speed and/or radial position of the mass and/or rotor. The command processor calculates a force generated by the mass as the mass and rotor rotates and the command processor manages electrical current to the electric motor. The monitor processor interprets data received from the second sensor and energizes the braking coil by closing the third relay when the data from the second sensor indicates an out of range condition for the rotational speed or position of the mass or rotor position. The monitor processor may also interpret data from the vibration sensor and energize the braking coil in response to the interpreted data.

### BRIEF DESCRIPTION OF THE DRAWINGS

FIG. 1 provides a block diagram of a prior art CFG system.

FIG. 2 provides a block diagram of one embodiment of the improved CFG system with the braking coil circuit inactive and the CFG system in a conventional operating mode.

FIG. 3 provides a perspective view of the primary interior components of an improved electric motor for use in the disclosed CFG systems.

FIG. 4 provides a front view of the primary interior components of an improved electric motor for use in the disclosed CFG systems.

FIG. 5 provides a cut-away view of the electric motor components taken at line 5 of FIG. 4.

FIG. 6 provides a cut-away view taken along line 6-6 of FIG. 5.

FIG. 7 is a sectional side view illustrating an exemplary mechanical assembly of a circular force generator. The embodiment of FIG. 7 does not include a braking coil.

FIG. 8 is an exploded perspective view illustrating an exemplary mechanical assembly of a circular force generator.

FIG. 9 is a partially-exploded perspective view illustrating a portion of the motor components of a circular force generator.

FIG. 10 provides a perspective view of the mass of the CFG installed on the rotor.

FIG. 11 generically depicts imbalance rotors of a circular force generator and the vector forces resulting from rotation of the imbalance rotors.

FIG. 12 illustrates force generation using a circular force generator with two corotating imbalanced rotors to create a circular force with controllable magnitude and phase, thereby providing a CFG.

FIG. 13 provides a block diagram of one embodiment of the improved CFG system with the braking coil circuit active, i.e. energized, and the CFG system in a disabled mode.

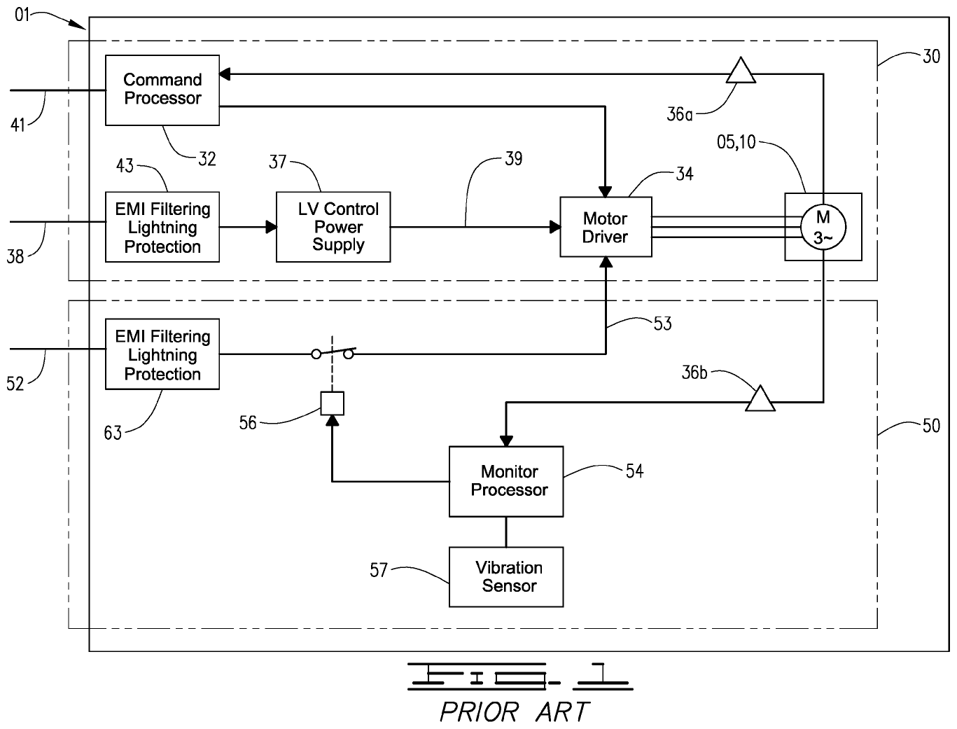

### DETAILED DESCRIPTION

The drawings included with this application illustrate certain aspects of the embodiments described herein. However, the drawings should not be viewed as exclusive embodiments. The subject matter disclosed is capable of considerable modifications, alterations, combinations, and equivalents in form and function, as will occur to those skilled in the art with the benefit of this disclosure.

The present disclosure may be understood more readily by reference to these detailed descriptions. For simplicity and clarity of illustration, where appropriate, reference numerals may be repeated among the different figures to indicate corresponding or analogous elements. The following description is not to be considered as limiting the scope of the embodiments described herein. The drawings are not necessarily to scale and the proportions of certain parts may have been exaggerated to better illustrate details and features of the present disclosure. Also, the phraseology and terminology employed herein is for the purpose of description and should not be regarded as limiting except where indicated as such.

Throughout this disclosure, the terms “about”, “approximate”, and variations thereof, are used to indicate that a value includes the inherent variation or error for the device, system, or measuring method being employed as recognized by those skilled in the art.

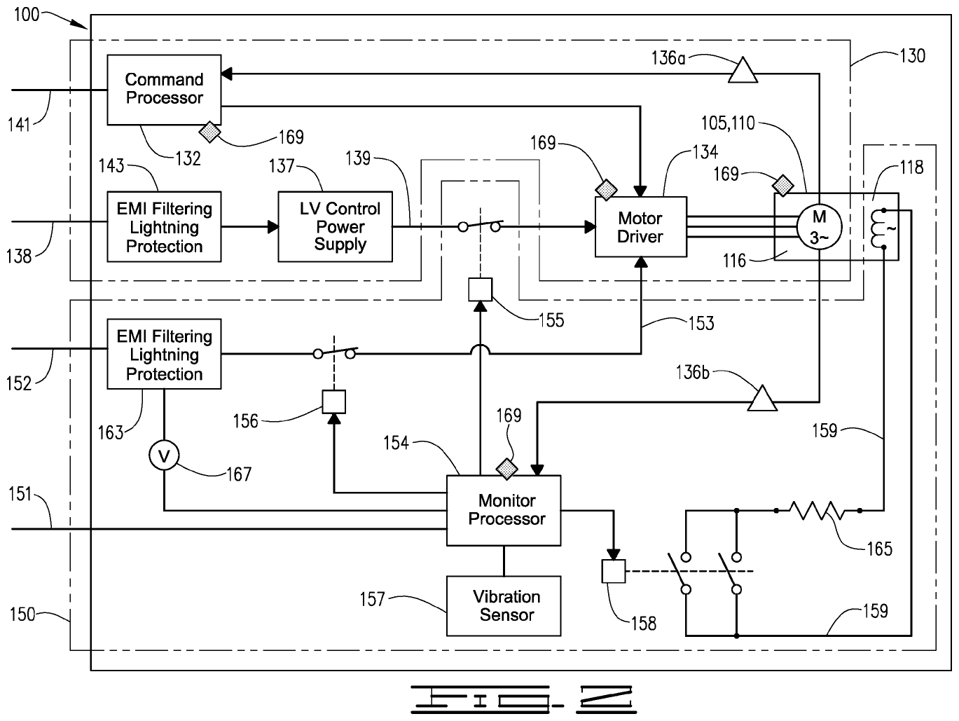

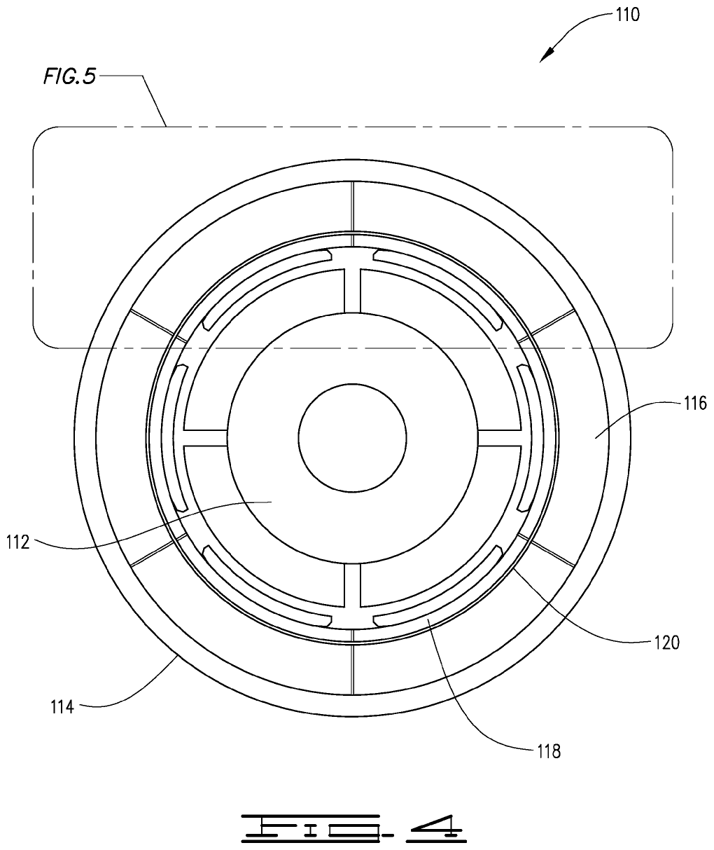

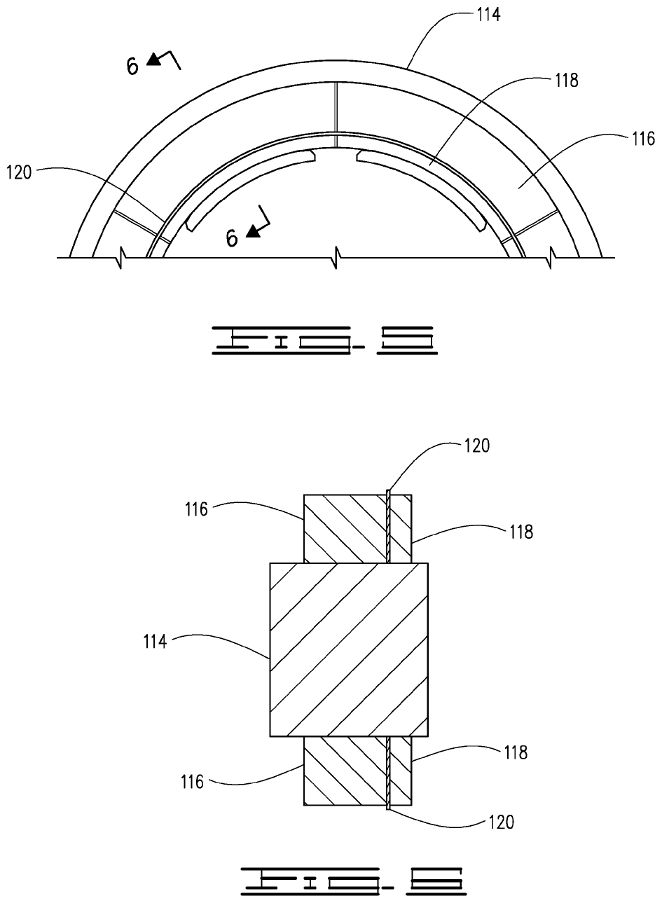

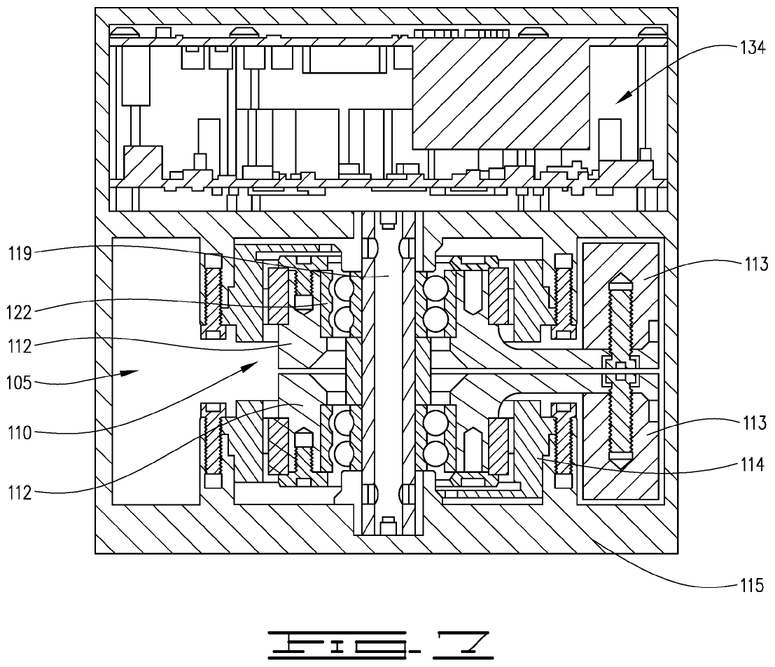

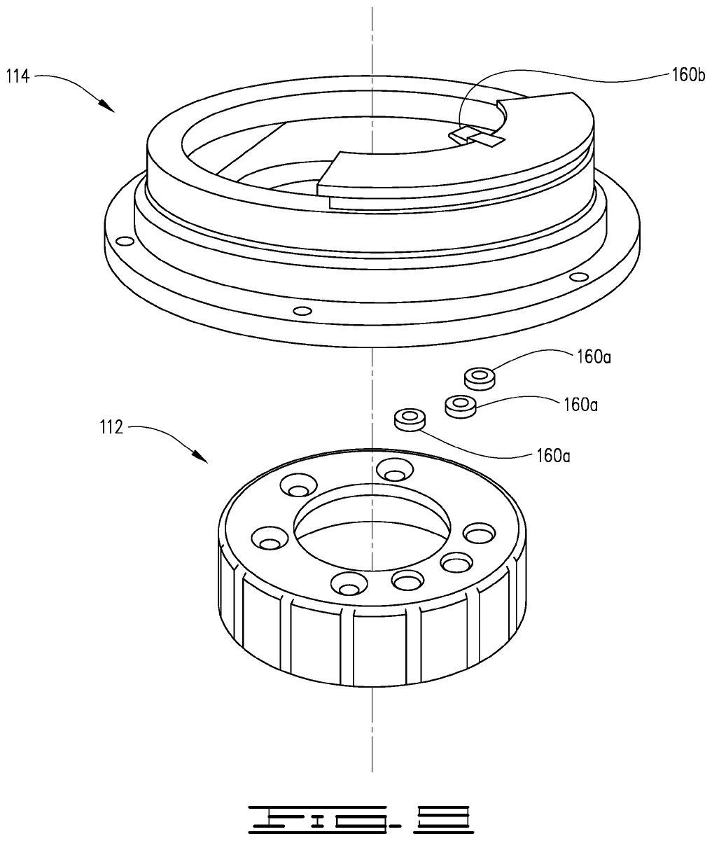

The improved CFG system 100 will be described with reference to FIGS. 2-12. CFG system 100 is suitable for use in connection with any structure or vehicle which would benefit from a reduction in structural vibrations.

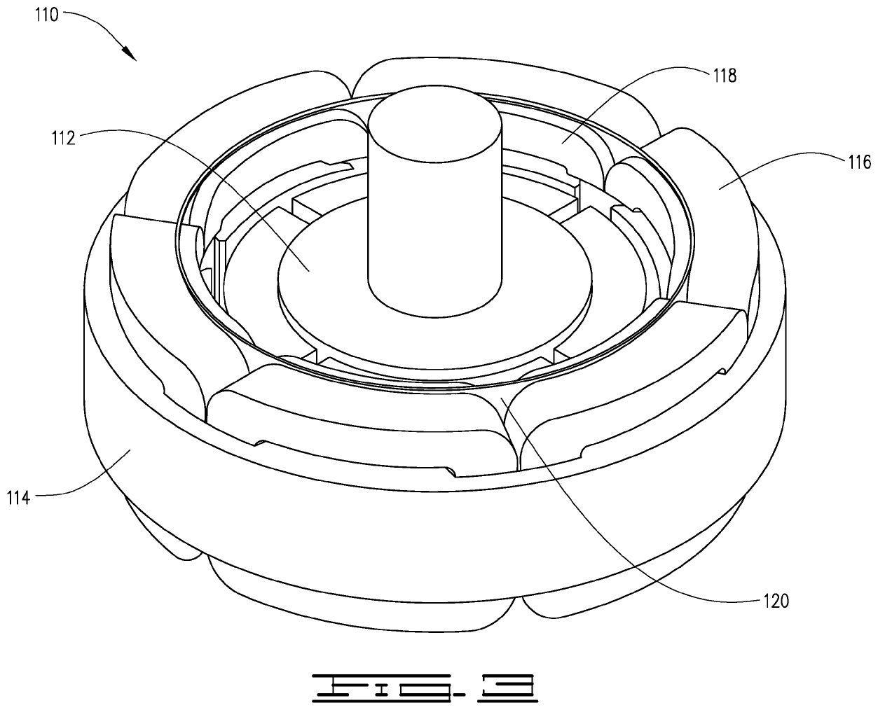

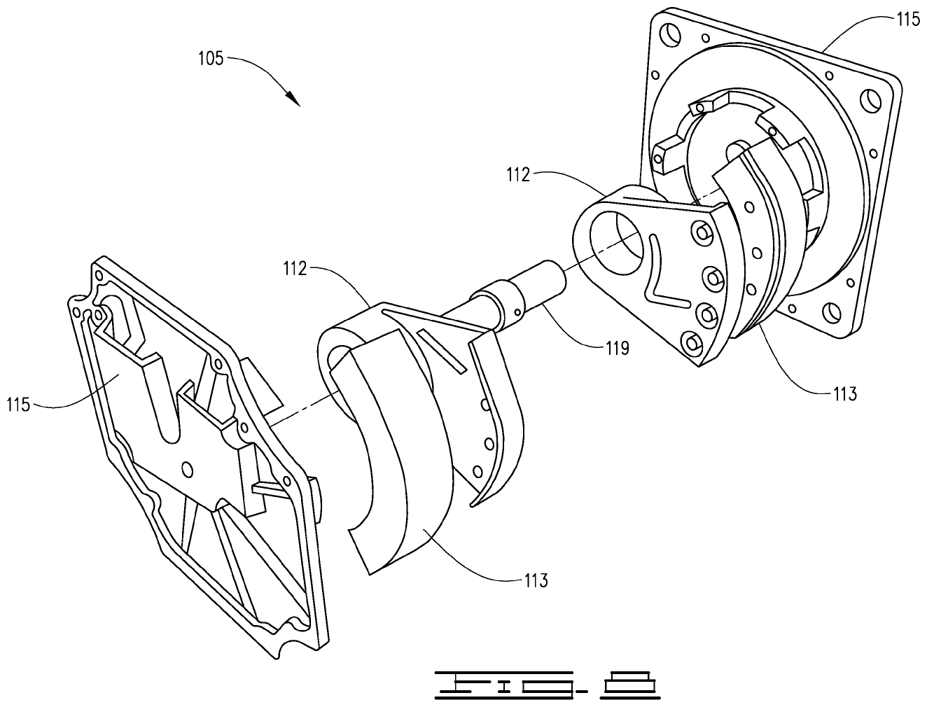

With reference to FIG. 2, CFG system 100 has three primary components: a circular force generator (CFG) mechanical assembly 105; a command circuit 130; and, a monitoring system 150. CFG mechanical assembly 105 may have multiple rotors 112 with each rotor supporting, directly or indirectly, a mass 113. See FIGS. 7-9. Command circuit 130 provides primary control over CFG mechanical assembly 105 while monitoring system 150 provides safety control of CFG mechanical assembly 105 including the ability to override command circuit 130. Thus, monitoring system 150 provides a failsafe during operation of CFG system 100. CFG mechanical assembly 105 includes at least one electric motor 110. In some embodiments, each electric motor 110 includes an integral braking coil 118 as described in more detail below with reference to FIGS. 3-6. In other embodiments with multiple motors 110, a single braking coil 118 will suffice.

FIGS. 7-9 depict an exemplary CFG mechanical assembly 105 without the incorporation of a braking coil 118. FIGS. 3-6 depict the improvement to electric motor 110 of CFG mechanical assembly 105 where the stator core 114 of at least one electric motor 110 has been modified by incorporation of a braking coil 118.

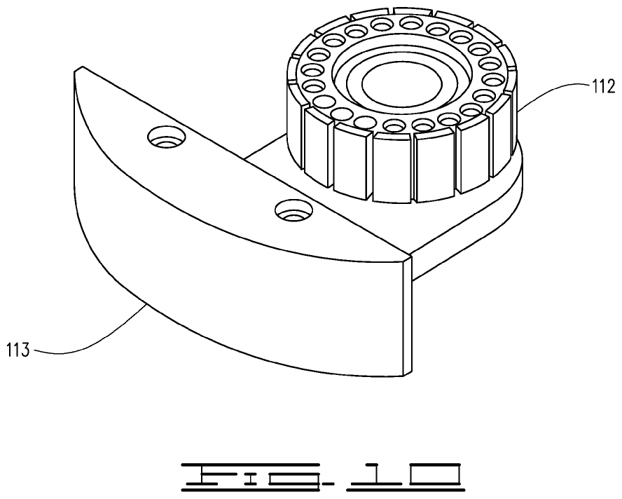

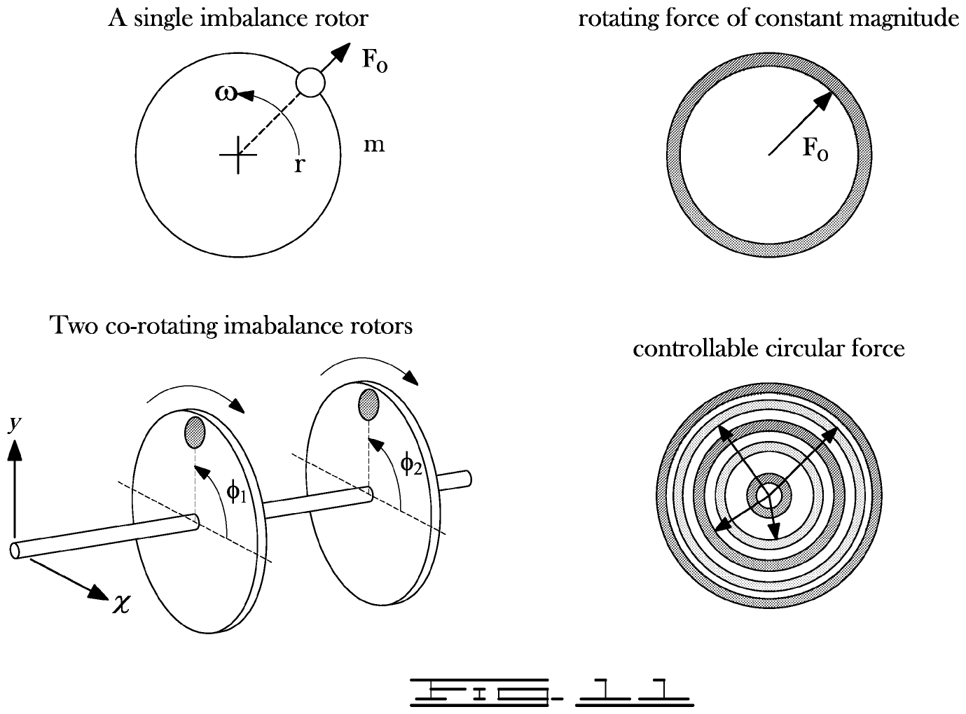

As depicted by FIGS. 7-10, a typical CFG mechanical assembly 105 includes one or more motors 110. Each motor 110 includes a stator core 114 and a rotor 112. In the embodiment depicted, stator core 114 is mounted to end plates 115. A rotor 112 of each electric motor 110 is coupled for rotation about a stationary center shaft 119 by a bearing 122 mounted inside the motor 110. A rotating mass 113 is eccentrically connected to each rotor 112 such that rotation of the rotor 112 about the shaft 119 can generate a “circular” force. CFG mechanical assembly 105 also includes a command processor 132 to monitor the rotational position of the rotating mass 113. In the embodiment depicted in FIGS. 7-10, speed/position sensors 136a, 136b take the form of a Hall sensor 160b which monitors the position of target magnets 160a. FIG. 9 depicts only the motor portion of rotor 112 as mass 113 has been omitted. FIG. 10 depicts mass 113 carried by rotor 112. The configuration shown in FIGS. 7-10 is but one exemplary arrangement, and the particular number and positioning of the rotors and sensors used in CFG mechanical assembly 105 can be modified based on a variety of design considerations for the environment of use. For example, rotor 112 may carry mass 113 directly or indirectly or rotor 112 may drive a separate rotor which directly or indirectly supports mass 113.

Command circuit 130 provides primary operational control over electric motor 110 thereby controlling the magnitude and phase and frequency of the force produced by CFG mechanical assembly 105. Command circuit 130 includes a force command digital bus 141, a command processor 132, speed and/or position sensors 136a, a control power source 138, electromagnetic interference (EMI) filter with lightning protection 143, a low voltage control power supply 137, primary windings 116 and a motor driver 134.

As depicted in FIG. 2, speed and/or position sensors 136a monitor operation of CFG mechanical assembly 105 and provide data to command processor 132. Command processor 132 also receives reference rotor 112 radial speed, force commands and force magnitude and phase data via force command digital bus 141. Typically, force magnitude and phase are calculated as described below using the radial position of rotor 112. The command processor 132 determines the desired position of each rotor 112 from the force command, and then compares this to the actual speed and rotor position measurements provided by 136a within a control loop that ensures that the actual rotor position and speed are close to the commanded position and speed so that the actual generated force is close to the command force with little error. Command processor 132 has been preprogrammed to interpret the received data and subsequently provide motor commands to motor driver 134. Motor driver 134 responds to the received commands by adjusting the voltage and current to electric motor 110 to alter the rotational speed and position of rotors 112 within CFG mechanical assembly 105 thereby managing the phase and force magnitude produced by CFG mechanical assembly 105.

As depicted in FIG. 2, during conventional operation of CFG system 100, electrical current passes from control power source 138 to EMI filter 143 and then to low voltage control power supply 137. Electric current from low voltage control power supply 137 passes through a closed relay 155 located in circuit 139 prior to reaching motor driver 134. Thus, opening of relay 155 provides the ability to remove low voltage power from motor drive 134. Likewise, the opening of relay 156 provides the ability to remove high voltage power to the motor drive 134. As depicted in FIG. 2, monitor processor 154 controls first and second relays 155 and 156. Thus, two different dissimilar means of removing power to the motor driver 134 (and motor 110) are provided for redundancy and safety. As used herein the term “relay” includes other electronic switching devices, including solid state relays and other devices for interrupting or completing electrical circuits. For consistency, this disclosure generally refers to “relay” throughout. Conventional operation of CFG system 100 refers to application of an input voltage from motor power 152 to motor driver 134 in a predetermined range depending on the type of CFG system 100. Most CFG systems utilize a 28 volt nominal or a 270 volt nominal supply to motor driver 134.

With continued reference to FIG. 2, CFG system 100 also includes monitoring system 150 which provides safety control of electric motor 110 and CFG mechanical assembly 105 by interrupting flow of electric supply voltage to motor driver 134 and electric motor 110 and providing electronic braking to the motor 110 through the braking coil 118 and circuit 159. Monitoring system 150 includes a second power supply identified as motor power 152, a monitor processor 154, a vibration sensor 157, speed and/or position sensors 136b, a braking coil 118 incorporated into electric motor 110 and a third relay 158 located within circuit 159. As depicted in FIG. 2, during operation of CFG system 100, third relay 158 is held open by monitor processor 154 as long as voltage into monitoring system 150 is above a predetermined level. Likewise, during operation of CFG system 100, relays 155 and 156 are held closed by monitor processor 154. In most instances, this voltage level will be determined by the operational condition of CFG system 100, i.e. the field of use. Since third relay 158 is held open during conventional operation of CFG system 100, a loss of voltage will result in closure of relay 158 and energization of circuit 159 while the same loss of voltage will result in opening of relays 155, 156.

As depicted, relay 158 includes a two pole switch operated by relay 158. However, relay 158 may take the form of solid state switching electronics or an electromechanical relay capable of energizing circuit 159, i.e. completing the circuit. The type of relay selected for relay 158 will depend on the environment of CFG system 100. Safety critical operations, e.g. aircraft environment, may dictate a two pole configuration. As described above, during conventional operation of CFG system 100, monitor processor 154 maintains relay 158 in an open position thereby precluding current within circuit 159. Thus, relay 158 does not provide for electric current flow unless actuated, i.e. closed, in response to monitor processor 154. As depicted in FIG. 13, operation of relay 158 completes circuit 159 resulting in a braking action due to rotation of rotor 112 within braking coil 118. Additional circuitry may be included to verify the continuity or other characteristics of the braking coil 118 and proper functionality of the brake control circuitry as part of unit self-test. The configuration of braking coil 118 is depicted in detail in FIGS. 3, 4 and 6.

As depicted in FIG. 2, motor power 152 also provides electric current to motor driver 134 via circuit 153. Electric current from motor power 152 passes through a second EMI filter with lightning protection 163. Located within circuit 153 between EMI filter 163 and motor driver 134 is a second relay 156. During conventional operation of CFG system 100, monitor processor 154 holds relay 156 in the closed position. As with relay 155, opening of relay 156 provides the ability to remove power from motor driver 134. Again the number and type of relays used to interrupt electric current to motor driver 134 will depend on the environment of use. FIG. 2 provides an exemplary embodiment of the circuitry used for controlling and managing the operation of CFG 100 including braking coil 118 with relays 155, 156 and 158 in position for conventional operation of CFG system 100. Other circuit embodiments are possible. For example, embodiment depicted in FIG. 2 is particularly useful on aircraft. As such, the embodiment utilizes two relays 155, 156 to provide redundant safety during aircraft operations. However, the current invention will have applicability to any structure requiring vibration cancelling. In those environments where redundancy is not required, monitoring system 150 will perform satisfactorily with a single relay positioned to remove power from motor driver 134. When redundancy is not a requirement, a single speed and/or position sensor 136a may replace the pair of speed and/or position sensors 136a, 136b. When using a single sensor 136a, appropriate electrical communication will be provided to both command processor 132 and monitor processor 154. Likewise, FIG. 2 depicts separate power sources 138 and 152 providing electric current to motor driver 134 through separate relays 155, 156. However, CFG 100 could be configured to use a single power source with internal splitting of the electrical circuit to accomplish the same operation. As a further option, relays 155 and 156 could be replaced by a single double pole relay or switch similar to relay 158.

In the embodiment of FIG. 2, monitor processor 154 receives data from speed and/or position sensors 136b and from vibration sensor 157. Monitor processor 154 has been preprogrammed to interpret the received data and subsequently provide operational control over relays 155, 156 and relay 158. In the event that CFG mechanical assembly 105 experiences a failure or an out-of-range condition as determined by data provided by vibration sensor 157 or sensor(s) 136b (vibration, speed, rotor position, CFG force which can be estimated from the speed and rotor position of all of the rotors), monitor processor 154 will interpret the received data and use internal programming to generate a command to direct the opening of relays 155, 156 and closing of relay 158. See FIG. 13. Thus, monitor processor 154 overrides command circuit 130 by removing power from motor driver 134.

Upon closing of relay 158, rotation of rotor 112 within braking coil 118 creates an opposing torque to rotation of rotor 112 due to induced eddy currents within braking coil 118. Thus, rotation of rotor 112 within braking coil 118 generates an electric current which is subject to the electrical resistance of circuit 159 and optional resistor 165. As a result, completion of circuit 159 by closure of relay 158 allows braking coil 118 to produce a braking action on rotor(s) 112. The resulting braking action rapidly brings rotor(s) 112 of CFG mechanical assembly 105 to a safe speed or optionally to a full stop. In most embodiments, full stop of rotor 112 is not required as reducing the angular velocity to a safe speed will generally alleviate the unsafe condition which triggered the closure of relay 158 and opening of relays 155, 156. Further, as rotor 112 decelerates to a safe speed, friction within the mechanical components will bring rotor(s) 112 to a full stop.

In the embodiment of FIG. 2, monitor processor 154 also communicates with external controller(s) (not show) via a two-way bus 151. Data exchanged via two-way bus 151 allows the external controller(s) to interpret data from monitor processor 154 and to direct monitor processor 154 to activate braking coil 118 by closing of relay 158 with the corresponding opening of relays 155, 156. Thus, activation of braking coil 118 and deactivation of electric motor 110 may occur in response to data received from the external controller(s). For example, when installed on an aircraft, the external controller may take the form of an override switch suitable for operation by an onboard crew member. Thus, monitor processor allows a crew member to temporarily disable CFG system 100.

FIGS. 3-6 depict the incorporation of braking coil 118 into electric motor 110. Electric motor 110 includes rotor(s) 112, a stator core 114 and primary windings 116. As previously indicated, rotor(s) 112 carry rotating masses 113 (shown only in FIGS. 7-9). Primary windings 116 have a traditional 3-phase configuration. Additionally, electric motor 110 has been modified to incorporate braking coil 118. The windings of braking coil 118 are positioned on the same stator core 114 as primary windings 116. As depicted in FIG. 3, braking coil 118 is positioned at the inner diameter of stator core 114 while primary windings 116 are located at the outer diameter of stator core 114. An electrically insulating material 120 physically separates braking coil 118 from primary windings 116 in each slot of stator core 114 to provide protection against a short between braking coil 118 and primary windings 116. Insulation layer 120 may be any suitable insulating material commonly used in electric motors. One example of an insulating material suitable for use as insulating layer 120 would be Nomex® 410 marketed by Dupont. Nomex® 410 is a family of insulating papers that provide high inherent dielectric strength, mechanical toughness, flexibility and resilience; however, any insulator commonly used in electric motors which provides the necessary dielectric protection will suffice.

Braking coil 118 is a single winding that occupies about 10% to about 50% of every slot in the stator. As a result incorporation of braking coil 118 into electric motor 110 does not significantly increase the overall size of electric motor 110. Additionally, braking coil 118 uses a wire gauge and conductor length sufficient to provide a braking action to rotor 112 following closure of relay 158 and completion of circuit 159. The wire gauge and length in combination with the rotation of rotor(s) 112 produces an electrical current within circuit 159. The resistance to electric current flow in circuit 159 and braking coil 118 is sufficient to overcome the inertial energy of spinning rotor(s) 112 such that rotor(s) 112 are brought to either a safe speed or a complete stop within a predetermined time upon closing of relay 158 and opening of relays 155 and 156. To limit the size of electric motor 110, the wire gauge and length is not sized to overcome the torque generated by primary windings 116 when relays 155 and 156 are closed, i.e. when primary windings 116 are energized.

The actual size of the electric motor 110, including braking coil 118, including wire gauge and length, will be determined by the size and mass of rotor(s) 112 and the operational environment of CFG system 100. In particular, braking coil 118 should be designed to use a wire gauge and length that will provide the necessary electrical resistance such that the resulting current produced by braking coil 118 following closure of relay 158 is at or below that value which will dissipate energy generated during a braking event as an acceptable level of heat. If necessary an optional supplemental resistor 165 may be incorporated into the braking coil circuit 159 to ensure adequate resistance, heat dissipation and operational limits of components within circuit 159. In some embodiments, the incorporation of additional resistors 165 or other electrical components within circuit 159 will limit the current within circuit 159 to a range of about 8 to 15 amps. In most embodiments approximately 10 amps will suffice. When used, resistor(s) 165 would be applied in series with braking coil 118. However, in some embodiments, the single wound wire of braking coil 118 will provide the braking torque required and the ability to dissipate the generated heat which occurs during the short duration, high energy stopping events. Selection of the gauge, wire length and overall size of braking coil 118 can be determined with knowledge of the inertial energy generated by rotor 112 during electric motor operation.

Thus, to provide the desired safe operation of CFG mechanical assembly 105 following a sensed failure or out of range condition, braking coil 118 must overcome the inertial torque generated by rotor(s) 112 and bring rotor(s) 112 to a safe speed or full stop in a period of time which precludes damage to CFG mechanical assembly 105 or the supporting structure. As noted above, braking coil 118 could be designed to brake a rotor 112 spinning at 30 Hz and reduce its speed to less than 5 Hz within 0.5 seconds. Braking torque, τ_{b}, is equal to:

$${τ}_{b} = {k}_{t} * i = {k}_{t} * ( ε / R )$$

Where kₜ is the torque constant for the braking coil 118, i is the current in braking coil 118, ε is the back electromotive force (emf), and R is the total resistance in the braking circuit (relay 158, circuit 159 and braking coil 118). When rotor 112 is spinning, a back emf (or “counter” emf) is generated in braking coil 118. Closing relay 158 essentially converts rotor(s) 112 and braking coil 118 into a generator causing current to flow through the resistive elements of circuit 159. As in any generator, the spinning rotor(s) 112 must overcome the resistance produced by eddy currents within braking coil 118 and electrical resistance within circuit 159 to continue rotating. In this instance, the resulting resistance dissipates the inertial torque of rotor(s) 112 resulting in the deceleration of rotor(s) 112. Note, that inductance in the braking circuit is not included in the calculations because it is typically an insignificant contributor. Torque can also be represented as a function of the back emf constant, k_{b}, and rotor speed, ω:

$${τ}_{b} = ( {k}_{t} * {k}_{b} * ω ) / R$$

The back emf decreases as the speed decreases, so the braking torque is reduced as the rotor slows down, i.e. electric current produced by braking coil 118 is reduced. Bearing friction brings rotor 112 to rest at these reduced speeds. Motor constants kₜ and k_{b} are characteristics of the braking coil design, dependent on the wire gage, number of turns, magnet strength, air gap to the rotor, length and diameter of motor 110, etc. Therefore, braking torque for a given motor is proportional to rotor speed and the resistance in the braking circuit. Higher braking torque, provided by increasing the number of coil turns and wire length or an increase in motor magnet strength, will decelerate the rotor more quickly. A low resistance value, R, reduces the duration of the brake event at the expense of higher current in the circuit. Note that inductance is again omitted for the aforementioned reason. Finally, selectively choosing the wire gauge in braking coil 118 can impact R, kₜ and k_{b} thereby allowing one to adjust the resulting current in braking coil 118 and circuit 159.

As depicted in FIG. 2 and discussed above, during conventional operation of CFG 100 relays 155 and 156 are held closed thereby powering motor driver 134 and electric motor 110 to drive CFG mechanical assembly 105. Braking coil 118 remains inactive as relay 158 is held open during conventional operation of CFG 100. Thus, during conventional CFG 100 operation, braking coil 118 and rotor 112 function as an electrical generator with no electrical load. Braking coil 118 generates an open circuit voltage with amplitude and frequency proportional to speed. However, an electrical current does not occur in braking coil 118 or circuit 159 because of the lack of an electrical load. As a result, braking coil 118 does not apply electromagnetic braking torque to rotor 112 during conventional operation of CFG mechanical assembly 105.

In most embodiments, a voltage sensor 167 provides an additional safeguard to the operation of CFG system. Voltage sensor 167 monitors voltage of the current from EMI filter 163 to monitor processor 154 and reports data concerning the voltage of the electrical current to monitor processor 154. In the event voltage sensor 167 reports a drop in voltage below a predetermined value to monitor processor 154, monitor processor 154 includes programming which will automatically begin a shutdown routine which includes energizing circuit 159 by closure of relay 158 while also overriding command circuit 130 by opening relays 155, 156. Typically, voltage to monitor processor 154 is in the range of about 1.1 Volts to about 3.6 Volts depending on processor input voltage requirements. If the voltage drops to within 5% of the lower desired limit then the automatic shutdown routine of monitor processor 154 is triggered. If the voltage exceeds the desired upper limit for a period of about 0.05 seconds, then the automatic shutdown routine of monitor processor 154 is triggered. Thus, when loss of monitoring system 150 appears to be likely due to improper voltage at monitor processor 154, the programming of monitor processor 154 provides for the safe shutdown of CFG system 100.

Likewise, the optional incorporation of temperature sensors 169 enhance the safe operation of CFG system 100. Both command circuit 130 and monitoring system 150 may include temperature sensors on key elements. For example, in command circuit 130, temperature sensors 169 are associated with command processor 132 and motor driver 134. In monitoring system 150, a temperature sensor 169 is associated with monitor processor 154. An additional temperature sensor may be associated with stator core 114 or electric motor 110. Each temperature sensor 169 is in data communication directly or indirectly with monitor processor 154. Monitor processor 154 is preprogrammed with the temperature ranges for each temperature sensor appropriate for the associated component and the operational condition of CFG system 100. If monitor processor 154 determines that the associated component has a sensed temperature outside of the predetermined range, then monitor processor 154 will energize circuit 159 by closure of relay 158 while also overriding command circuit 130 by opening relays 155, 156. Additional temperature sensors 169 with the same capabilities may be incorporated into CFG system 100 as needed to provide for safe shutdown of CFG system 100.

The ability of CFG system 100 to ensure safe operation of CFG mechanical assembly 105 will be described with reference to FIG. 2. As reflected by FIG. 2, the ability of monitoring system 150 to override command circuit 130 provides the desired safety control over CFG mechanical assembly 105. During operation of CFG system 100, monitor processor 154 continuously receives data from vibration sensor 157 or speed/position sensors 136b and interprets this data using preprogrammed software to determine if a fault condition exists. (As previously noted, in situations that do not require redundancy, sensors 136a can be adapted for use in monitoring system 150.) Upon detection of a fault condition that warrants disabling CFG mechanical assembly 105 or upon receipt of an override command from an external source, monitor processor 154 directs the opening of relays 155 and 156 and the closing of relay 158. See FIG. 13. The opening of relays 155 and 156 prevents motor driver 134 from controlling motor 110 by removing electric current from both motor driver 134 and motor 110. Thus, motor 110 starts to freewheel upon removal of power from motor driver 134. The closing of relay 158 completes circuit 159 and allows the interaction of rotor 112 with braking coil 118 to produce an electric current within circuit 159. As discussed above, the production of electric current 159 produces a resistance to rotation of rotor(s) 112 associated with braking coil 118. Optionally, monitor processor 154 may be programmed to provide a time delay between determination of a fault that necessitates shut down of CFG 100 and the actual implementation of the shutdown process. This programmed delay allows the monitor processor 154 the option of determining if the detected fault is temporary in nature.

As noted above, closing of relay 158 energizes circuit 159 and applies an electrical load to braking coil 118. During this time, braking coil 118 and rotor 112 continue functioning as an electrical generator. However, with an electrical load present, braking coil 118 and rotor 112 provide power to circuit 159 by converting kinetic energy to power that is dissipated as heat energy created when electrical current flows through an electrical resistance. In the example provided by FIG. 2, the electrical current flowing in braking coil 118 is a function of the rotor speed, the braking coil motor constant, the braking coil winding resistance, and any additional resistance present in the brake control circuitry of monitoring system 150, i.e. elements within circuit 159 such as relay 158, optional resistor(s) 165 and braking coil 118. The electromagnetic braking torque applied to the rotor 112 is proportional to the electrical current flowing in braking coil 118 as determined by the formulas above. While greater electrical current results in greater torque and faster deceleration, the increased electrical and thermal stresses on relay 158, circuit 159 and braking coil 118 must be considered as limiting factors. As noted above, the winding resistance of braking coil 118 may be sufficient to limit the electrical current; however, adding an optional resistor 165 in circuit 159 in series with braking coil 118 to further limit current may be desired.

As described above, the default state for relays 155 and 156 is in the open position. Likewise, the default state for relay 158 is the closed position, as depicted in FIG. 13. Thus, during normal operations voltage supplied to command circuit 130 and monitor system 150 ensures that relays 155 and 156 are held in the closed position while relay 158 is held in the open position, as depicted in FIG. 2. Thus, loss of electrical power from motor power 152 to monitoring system will result in CFG system 100 reverting to a safe mode as depicted in FIG. 13.

Thus, CFG system 100 and the method of operating CFG system 100 provides the ability to reduce the rotational speed of rotors 112 to a safe range in response to sensed or calculated unsafe conditions. The sensed unsafe conditions may be determined by monitor processor 154 upon interpretation of data received from sensors 136b and vibration sensor 157. These conditions may include one or more of overspeed or underspeed of rotor(s) 112, incorrect rotor 112 position, excessive vibrations or the calculated CFG force or phase of the produced CFG force determined to be out of range. An out of range condition for any of these factors may be identified by monitor processor 154 as a basis for overriding command system 130 and energizing circuit 159.

As known to those skilled in the art, CFG force and phase can be calculated using rotor speed and position. For example, index pulses produced by sensors 136a or 136b can be used to provide an estimate of the speed and phase position for each rotor, which can then be used to estimate the force. Equation (1) below illustrates how to compute the force of one rotor.

$$F = mr {ω}^{2} cos ( ω t + φ ) \qquad Eq ( 1 )$$

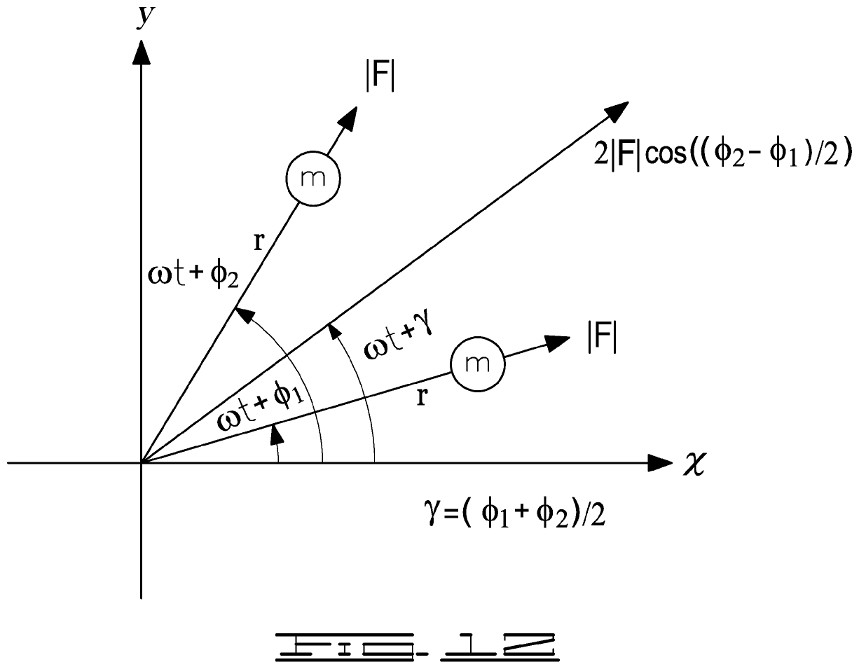

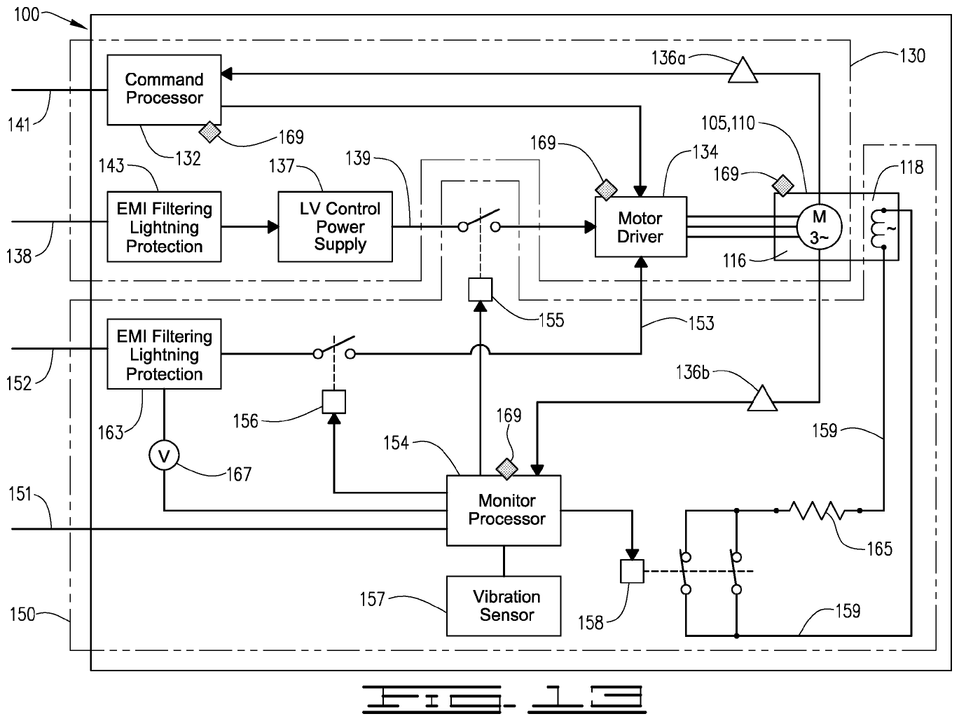

In Equation 1 m is the mass of mass 113, r is the radius of rotation of mass 113, ω is the rotational speed, t is time and φ is rotational phase position of mass 113. FIG. 11 provides a visual representation of the forces produced by a CFG. Determination of CFG force and phase as being out of range may occur within preprogrammed monitor processor 154 or by another preprogrammed controller located outside of monitoring system 150 but in communication with monitor processor via two-way command bus 151. FIG. 12 illustrates a way to compute the force output of 2 rotors (a CFG). In FIG. 11 phase q of a first imbalanced mass within the CFG with respect to a second imbalanced mass within the CFG (i.e., the relative phase) determines the magnitude of resultant rotating force vector. The resulting force in a zero-force case and a full-force case of imbalance masses of the CFG is illustrated in FIG. 12. In the zero-force case the relative phase φ₂−φ₁ is 180 degrees and resulting force rotating vector has a magnitude of zero. In the full-force case, the relative phase φ₂−φ₁ is 0 degrees and resulting rotating force vector has a maximum magnitude of 2|F|. For relative phases φ₂−φ₁ between 0 and 180 degrees, the magnitude of resulting rotating force vector will be between zero and the maximum. The collective phase γ of rotating force vector can be varied to provide phasing between CFGs. Through control of phase ϕ of each imbalance mass in the CFG has the magnitude and absolute phase of the rotating force vector produced by CFG can be controlled.

Thus, the present invention includes a method for safely shutting down CFG system 100. The method utilizes monitoring system 150 to identify an out-of-range criteria as described above which necessitates overriding command circuit 130 and bringing rotor 112 to a safe speed. In this method data from at least one speed and/or position sensor 136a or 136b provides data to monitor processor 154. Additionally, vibration sensor 157 provides data concerning vibrations within the structure supporting CFG system 100. If monitor processor 154 determines the existence of an out-of-range condition for any of the monitored conditions, then monitor processor energizes circuit 159 by closing relay 158 while overriding command circuit by opening relays 155 and 156. As discussed above, the resulting electrical current in circuit 159 creates an opposing torque to the rotation of rotor 112 slowing rotor 112 to a safe speed. Below a certain speed, friction within CFG mechanical assembly 105 will bring rotor 112 to a stop. Optionally, a manual override may be provided to an operator of the structure supporting CFG system 100 such that manual shut down may be performed without waiting on monitor processor 154 to override command circuit 130. Manual shut down may be performed by the operator of the structure supporting CFG system 100 by sending a direct command to monitor processor 154 via circuit 151 or by removing power provided by motor power 152 from monitoring system 150.

Other embodiments of the present invention will be apparent to one skilled in the art. As such, the foregoing description merely enables and describes the general uses and methods of the present invention. Accordingly, the following claims define the true scope of the present invention.

1. A circular force generator system comprising:
an electric motor, the electric motor having a rotor, a stator core, primary windings and a braking coil;
a mass supported by the rotor;
a command circuit, the command circuit comprising:
a command processor;
a motor driver in electronic communication with the command processor, the motor driver providing electrical current to the primary windings;
a first sensor for monitoring rotational speed and/or radial position of the mass or rotor, the sensor in electronic communication with the command processor;
a first power supply, the first power supply providing electrical current to the motor driver via a first electrical circuit;
a monitoring system, the monitoring system comprising:
a monitor processor, the monitor processor controlling a first relay, the first relay positioned within the first electrical circuit, wherein the monitor processor provides control over the first relay such that opening of the first relay by the monitor processor removes electrical current from the electric motor driver;
a second relay, the second relay positioned within a second electrical circuit, wherein the monitor processor provides control over the second relay and closure of the second relay activates the braking coil.

2. The circular force generator system of claim 1, wherein the braking coil comprises:
a braking coil winding supported by the stator core, the winding forming a second distinct winding on the stator core from the primary windings;
an insulator separating the winding of the braking coil from the primary windings.

3. The circular force generator system of claim 2, wherein the braking coil winding has a length and gauge sufficient to induce an electrical current in the second electrical circuit upon closure of the second relay.

4. The circular force generator system of claim 1, wherein upon closure of the second relay the second electrical circuit has an electrical resistance and wherein upon closure of the second relay a resistance provided by braking coil winding when combined with the resistance of the second electrical circuit creates a braking action on the rotor of the electric motor.

5. The circular force generator system of claim 1,
wherein upon closure of the second relay the second electrical circuit has an electrical resistance and
wherein upon closure of the second relay the braking coil winding has an electrical resistance; and,
the resistance of the second electrical circuit combined with the resistance of the braking coil creates a braking action on the rotor of the electric motor sufficient to overcome an inertial energy produced by spinning of the rotor the braking action sufficient to bring the rotor to a safe rotational speed upon detection of a fault by the monitor processor.

6. (canceled)

7. (canceled)

8. The circular force generator system of claim 1, further comprising a vibration sensor, the vibration sensor in electronic communication with the monitor processor, the monitor processor is programmed to control the first and second relays in response to data received from the vibration sensor; and,
wherein the command processor is programmed to control the motor driver in response to the first sensor, the monitor processor is programmed to control the first and second relays in response to data received from the first sensor and to control the first and second relays in response to data received from an external source.

9. The force generator system of claim 1, wherein the monitoring system further comprises:
a second power supply, the second power supply providing electrical current to the motor driver via a third electrical circuit;
a third relay positioned within the third electrical circuit, the third relay is controlled by the monitor processor such that opening of the third relay removes electrical current from the motor driver.

10. (canceled)

11. (canceled)

12. (canceled)

13. (canceled)

14. The force generator system of claim 1, wherein the monitor processor is programmed to interpret data received from the sensor, from an external source or from a vibration sensor and to override operation of the command circuit when any one of the following fault conditions are determined: overspeed of rotor, underspeed of rotor, rotor position outside of a predetermined range, magnitude out of a predetermined range or vibrations in excess of a predetermined value.

15. The circular force generator system of claim 1, further comprising:
a second sensor for monitoring rotational speed and/or radial position of the mass or rotor, the second sensor in electronic communication with the monitor processor.

16. (canceled)

17. (canceled)

18. (canceled)

19. (canceled)

20. (canceled)

21. (canceled)

22. The circular force generator system of claim 15, further comprising a vibration sensor, the vibration sensor in electronic communication with the monitor processor, the monitor processor is programmed to control the first and second relays in response to data received from the vibration sensor; and,
wherein the command processor is programmed to control the motor driver in response to the first sensor and the monitor processor is programmed to control the first and second relays in response to data received from the second sensor and to control the first and second relays in response to data received from an external source.

23. (canceled)

24. (canceled)

25. (canceled)

26. (canceled)

27. (canceled)

28. (canceled)

29. A circular force generator system comprising:
an electric motor (110), the electric motor having a rotor, a stator core, primary windings and a braking coil;
a mass supported by the rotor;
a command circuit, the command circuit comprising:
a command processor;
a motor driver in electronic communication with the command processor, the motor driver providing electrical current to the primary windings;
a first sensor for monitoring rotational speed and/or radial position of the mass or rotor, the sensor in electronic communication with the command processor;
a first power supply, the first power supply providing electrical current to the motor driver via a first electrical circuit;
a monitoring system, the monitoring system comprising:
a second power supply, the second power supply providing electrical current to the motor driver via a second electrical circuit;
a monitor processor, the monitor processor controlling a first relay, the first relay positioned within the first electrical circuit, the monitor processor controlling a second relay, the second relay positioned within the second electrical circuit, wherein the monitor processor provides control over the first and second relays such that opening of the first and second relays by the monitor processor removes electrical current from the motor driver;
a second sensor for monitoring rotational speed and/or radial position of the mass or rotor, the second sensor in electronic communication with the monitor processor;
a third relay, the third relay positioned within a third electrical circuit, wherein the monitor processor provides control over the third relay and closure of the third relay activates the braking coil.

30. The circular force generator system of claim 29, wherein the braking coil comprises:
a braking coil winding supported by the stator core, the winding forming a second distinct winding on the stator core from the primary windings; and,
an insulator separating the winding of the braking coil from the primary windings.

31. The circular force generator system of claim 30, wherein the braking coil winding has a length and gauge sufficient to induce an electrical current in the third electrical circuit upon closure of the third relay.

32. The circular force generator system of claim 29,
wherein upon closure of the third relay the third electrical circuit has an electrical resistance;
wherein upon closure of the third relay the braking coil has an electrical resistance; and,
the resistance of the third electrical circuit combined with the resistance of the braking coil creates a braking action on the rotor of the electric motor sufficient to overcome an inertial energy produced by spinning of the rotor, the braking action sufficient to bring the rotor to a safe rotational speed upon detection of a fault by the monitor processor.

33. (canceled)

34. (canceled)

35. The circular force generator system of claim 29, further comprising a vibration sensor in electronic communication with the monitor processor, the monitor processor is programmed to control the first and second relays in response to data received from the vibration sensor; and,
wherein the command processor is programmed to control the motor driver in response to the first sensor, the monitor processor is programmed to control the first, second and third relays in response to data received from the second sensor and to control the first and second relays in response to data received from an external source.

36. (canceled)

37. (canceled)

38. (canceled)

39. (canceled)

40. The force generator system of claim 29, wherein the monitor processor is programmed to interpret data received from the second sensor, from an external source or from a vibration sensor and to override operation of the command circuit when any one of the following fault conditions are determined: overspeed of rotor, underspeed of rotor, rotor position outside of a predetermined range, magnitude out of a predetermined range or vibrations in excess of a predetermined value.

41. A method for controlling a force generator system, the method comprising:
providing a force generator system comprising:
an electric motor, the electric motor having a rotor, a stator core, primary windings and a braking coil;
a mass supported by the rotor;
a command circuit, the command circuit comprising:
a command processor;
a motor driver in electronic communication with the command processor, the motor driver providing electrical current to the primary windings;
a first sensor for monitoring rotational speed and/or radial position of the mass or rotor, the sensor in electronic communication with the command processor;
a first power supply, the first power supply providing electrical current to the motor driver via a first electrical circuit;
a monitoring system, the monitoring system comprising:
a monitor processor, the monitor processor controlling a first relay, the first relay positioned within the first electrical circuit, the monitor processor controlling a second relay, the second relay positioned within a second electrical circuit, wherein the monitor processor provides control over the first and second relays such that opening of the first and second relays by the monitor processor removes electrical current from the motor driver;
a second sensor for monitoring rotational speed and/or radial position of the mass or rotor, the second sensor in electronic communication with the monitor processor;
a third relay, the third relay positioned within a third electrical circuit, wherein the monitor processor provides control over the second relay and closure of the second relay activates the braking coil;
operating the force generator system by rotating the rotor and mass while using the first and second sensors to monitor rotational speed and/or radial position of the mass and/or rotor, the command processor calculating a force generated by the mass as the mass and rotor rotates and the command processor managing electrical current to the electric motor;
using the monitor processor to interpret data received from the second sensor;
energizing the braking coil when the data from the second sensor indicates an out of range condition for the rotational speed or position of the mass or rotor position.

42. The method for controlling a force generator system of claim 41, wherein the step of energizing the braking coil further includes the steps of opening the first relay and closing the second relay.

43. The method for controlling a force generator system of claim 41, wherein the step of energizing the braking coil induces an electric current in the third electrical circuit wherein the induced current creates an opposing torque to the rotation of the rotor thereby slowing the rotation of the rotor.

44. The method for controlling a force generator system of claim 41, wherein the monitoring system includes a vibration sensor, the vibration sensor in electronic communication with the monitor processor, and further comprises the step of:
the vibration sensor monitoring a vehicle which supports the circular force generator system for vibrations;
the vibration sensor providing vibration data to the monitor processor, the monitor processor programmed with an upper limit for vibrations; and,
energizing the braking coil when the monitor processor determines that vibrations exceed the upper limit.

45. The method for controlling a force generator system of claim 41, wherein the monitor processor interprets data received from the second sensor and determines if the force generated by the rotation of the mass is within a predetermined range of speed, phase and magnitude, the monitor processor energizing the braking coil upon determining that any one of speed, phase or magnitude are outside of the predetermined range.

46. A method for controlling a force generator system, the method comprising:
providing a force generator system comprising:
an electric motor, the electric motor having a rotor, a stator core, primary windings and a braking coil;
a mass supported by the rotor;
a command circuit, the command circuit comprising:
a command processor;
a motor driver in electronic communication with the command processor, the motor driver providing electrical current to the primary windings;
a first sensor for monitoring rotational speed and/or radial position of the mass or rotor, the sensor in electronic communication with the command processor;
a first power supply, the first power supply providing electrical current to the motor driver via a first electrical circuit;
a monitoring system, the monitoring system comprising:
a monitor processor, the monitor processor controlling a first relay, the first relay positioned within the first electrical circuit, wherein the monitor processor provides control over the first relay such that opening of the first relay by the monitor processor removes electrical current from the motor driver;
a second sensor for monitoring rotational speed and/or radial position of the mass or rotor, the second sensor in electronic communication with the monitor processor;
a second relay, the second relay positioned within a second electrical circuit, wherein the monitor processor provides control over the second relay and closure of the second relay activates the braking coil;
operating the force generator system by rotating the rotor and mass while using the first and second sensors to monitor rotational speed and/or radial position of the mass and/or rotor, the command processor calculating a force generated by the mass as the mass and rotor rotates and the command processor managing electrical current to the motor;
using the monitor processor to interpret data received from the second sensor;
energizing the braking coil when the data from the second sensor indicates an out of range condition for the rotational speed or position of the mass or rotor position.

47. The method for controlling a force generator system of claim 46, wherein the step of energizing the braking coil further includes the steps of opening the first relay and closing the second relay.

48. The method for controlling a force generator system of claim 46, wherein the step of energizing the braking coil induces an electric current in the second electrical circuit wherein the induced current creates an opposing torque to the rotation of the rotor thereby slowing the rotation of the rotor.

49. The method for controlling a force generator system of claim 46, wherein the monitoring system includes a vibration sensor, the vibration sensor in electronic communication with the monitor processor, and further comprises the step of:
the vibration sensor monitoring a vehicle which supports the circular force generator system for vibrations;
the vibration sensor providing vibration data to the monitor processor, the monitor processor programmed with an upper limit for vibrations; and,
energizing the braking coil when the monitor processor determines that vibrations exceed the upper limit.

50. The method for controlling a force generator system of claim 46, wherein the monitor processor interprets data received from the second sensor and determines if the force generated by the rotation of the mass is within a predetermined range of speed, phase and magnitude, the monitor processor energizing the braking coil upon determining that any one of speed, phase or magnitude are outside of the predetermined range.

51. A method for controlling a force generator system, the method comprising:
providing a force generator system comprising:
an electric motor, the electric motor having a rotor, a stator core, primary windings and a braking coil;
a mass supported by the rotor;
a command circuit, the command circuit comprising:
a command processor;
a motor driver in electronic communication with the command processor, the motor driver providing electrical current to the primary windings;
a first sensor for monitoring rotational speed and/or radial position of the mass or rotor, the sensor in electronic communication with the command processor;
a first power supply, the first power supply providing electrical current to the motor driver via a first electrical circuit;
a monitoring system, the monitoring system comprising:
a second power supply, the second power supply providing electrical current to the motor driver via a second electrical circuit;
a monitor processor, the monitor processor controlling a first relay, the first relay positioned within the first electrical circuit, the monitor processor controlling a second relay, the second relay positioned within the second electrical circuit, wherein the monitor processor provides control over the first and second relays such that opening of the first and second relays by the monitor processor removes electrical current from the motor driver;
a second sensor for monitoring rotational speed and/or radial position of the mass or rotor, the second sensor in electronic communication with the monitor processor;
a third relay, the third relay positioned within a third electrical circuit, wherein the monitor processor provides control over the third relay and closure of the third relay activates the braking coil;
operating the force generator system by rotating the rotor and mass while using the first and second sensors to monitor rotational speed and/or radial position of the mass and/or rotor, the command processor calculating a force generated by the mass as the mass and rotor rotates and the command processor managing electrical current to the electric motor;
using the monitor processor to interpret data received from the second sensor;
energizing the braking coil by closing the third relay when the data from the second sensor indicates an out of range condition for the rotational speed or position of the mass or rotor position.

52. The method for controlling a force generator system of claim 51, wherein the step of energizing the braking coil further includes the steps of opening the first and second relays and closing the third relay.

53. The method for controlling a force generator system of claim 51, wherein the step of energizing the braking coil induces an electric current in the second electrical circuit wherein the induced current creates an opposing torque to the rotation of the rotor thereby slowing the rotation of the rotor.

54. The method for controlling a force generator system of claim 51, wherein the monitoring system includes a vibration sensor, the vibration sensor in electronic communication with the monitor processor, and further comprises the step of:
the vibration sensor monitoring a vehicle which supports the circular force generator system for vibrations;
the vibration sensor providing vibration data to the monitor processor, the monitor processor programmed with an upper limit for vibrations; and,
energizing the braking coil when the monitor processor determines that vibrations exceed the upper limit.

55. The method for controlling a force generator system of claim 51, wherein the monitor processor interprets data received from the second sensor and determines if the force generated by the rotation of the mass is within a predetermined range of speed, phase and magnitude, the monitor processor energizing the braking coil upon determining that any one of speed, phase or magnitude are outside of the predetermined range.

56. The circular force generator system of claim 9, further comprising:
at least one temperature sensor, the temperature sensor associated with a component selected from the group consisting of: command processor, motor driver, monitor processor, stator core or electric motor;
wherein the temperature sensor is in data communication with the monitor processor.

57. The method for controlling a force generator system of claim 51, wherein the force generator system further comprises:
at least one temperature sensor in data communication with the monitor processor, the temperature sensor associated with a component selected from the group consisting of: command processor, motor driver, monitor processor, stator core or electric motor; and,
further comprising the step of energizing the braking coil when the monitor processor determines that the data provided by the temperature sensors indicates a sensed temperature outside of a predetermined range.

58. The circular force generator system of claim 1, further comprising:
a voltage sensor, the voltage sensor positioned to monitor the voltage of the electrical current to the monitor process and provide voltage data to the monitor processor;
the monitor processor programmed to begin a shutdown routine which includes energizing the braking coil when voltage to the monitor processor as determined by the voltage sensor drops to within 5% of a predetermined lower limit or exceeds a predetermined upper limit for at least 0.05 seconds.

59. The method for controlling a force generator system of claim 46, further comprising:
providing a voltage sensor to monitor the voltage of the electric current provided to the monitor processor, the voltage sensor in data communication with the monitor processor; and,
using the voltage sensor to report voltage data to the monitor processor;
if the voltage sensor reports a voltage drop to within 5% of a predetermined lower limit or a voltage which exceeds a predetermined upper limit for at least 0.05 seconds, then the monitor processor begins a shutdown routine thereby overriding the command circuit and energizing the braking coil.

60. The method for controlling a force generator system of claim 46, further comprising:
providing a voltage sensor to monitor the voltage of the electric current provided to the monitor processor, the voltage sensor in data communication with the monitor processor; and,
using the voltage sensor to report voltage data to the monitor processor;
if the voltage sensor reports a voltage drop to within 5% of a predetermined lower limit or a voltage which exceeds a predetermined upper limit for at least 0.05 seconds, then the monitor processor begins a shutdown routine thereby overriding the command circuit and energizing the braking coil.

61. The method for controlling a force generator system of claim 46, wherein the monitor system further comprises a second power supply, the second power supply providing electrical current to the motor driver via a third electrical circuit and wherein the monitor processor controls a third relay, the third relay positioned within the third electrical circuit, wherein the monitor processor provides control over the first and third relays such that opening of the first and third relays by the monitor processor removes electrical current from the motor driver.

62. The method for controlling a force generator system of claim 61, wherein the step of energizing the braking coil further includes the step of opening the third relay.

63. The circular force generator system of claim 29, further comprising:
at least one temperature sensor, the temperature sensor associated with a component selected from the group consisting of: command processor, motor driver, monitor processor, stator core or electric motor;
wherein the temperature sensor is in data communication with the monitor processor.

64. The circular force generator system of claim 29, further comprising:
a voltage sensor, the voltage sensor positioned to monitor the voltage of the electrical current to the monitor process and provide voltage data to the monitor processor;
the monitor processor programmed to begin a shutdown routine which includes energizing the braking coil when voltage to the monitor processor as determined by the voltage sensor drops to within 5% of a predetermined lower limit or exceeds a predetermined upper limit for at least 0.05 seconds.

65. A circular force generator system comprising:
an electric motor, the electric motor having a rotor, primary windings and a braking coil;
a mass supported by the rotor;
a command circuit, configured to control the primary windings of the electric motor; and,
a monitoring system, the monitoring system comprising:
a first relay, the first relay configured to deactivate the electric motor by removing power from the primary windings;
a second relay, the second relay configured to activate the braking coil.

66. The circular force generator system of claim 65, wherein the first relay is configured in a normally closed state during operation of the electric motor and configured for the open state when removing power from the primary windings and the second relay is configured in a normally open state and configured in the closed state to activate the braking coil.

67. The circular force generator system of claim 65, wherein the braking coil comprises:
a braking coil winding, the braking coil winding forming a second distinct winding from the primary windings;
an insulator separating the winding of the braking coil from the primary windings.

68. The circular force generator system of claim 67, wherein the braking coil winding has a length and gauge sufficient to induce an electrical current in the second electrical circuit upon closure of the second relay.

69. A method for controlling a force generator system, the method comprising:
providing a force generator system comprising:
an electric motor, the electric motor having a rotor, primary windings and a braking coil;
a mass supported by the rotor;
a command circuit, the command circuit configured to control the primary windings of the electric motor;
a monitoring system, the monitoring system comprising:
a first relay (155), the first relay deactivates the electric motor by removing power from the primary windings;
a second relay (158), the second relay activates the braking coil;
operating the force generator system by rotating the rotor while monitoring rotational speed and/or radial position of the rotor;
calculating a force generated by the rotor as the rotor rotates;
energizing the braking coil when an out of range condition for the rotational speed or position of the rotor exists.

70. The method for controlling a force generator system of claim 69, wherein the step of energizing the braking coil further includes the steps of opening the first relay and closing the second relay.
